(19) United States
(12) Patent Application Publication
(10) Pub. No.: US 2020/0000998 A1
(43) Pub. Date: Jan. 2, 2020

(54) A VENOVENOUS ECMO (EXTRACORPOREAL MEMBRANE OXYGENATION) CANNULA

(71) Applicant: T.C ISTANBUL MEDIPOL ÜNIVERSITESI, Istanbul (TR)

(72) Inventors: Ece SALIHOGLU, Istanbul (TR); Yahya YILDIZ, Istanbul (TR)

(21) Appl. No.: 16/484,552

(22) PCT Filed: Feb. 12, 2018
(86) PCT No.: PCT/TR2018/000010

§ 371 Date: Aug. 8, 2019

(30) Foreign Application Priority Data
Feb. 15, 2017 (TR) 2017/02278

(51) Int. Cl. A61M 1/36; A61M 1/14; A61L 29/10; A61L 29/16

(52) U.S. Cl. CPC A61M 1/3659; A61M 1/14; A61M 25/0045; A61L 29/16; A61L 29/106

(57) ABSTRACT

This invention is related to a dual lumen triple brimmed venovenous ECMO (Extracorporeal Membrane Oxygenation) cannula (1).

### TECHNICAL FIELD

This invention is related to a dual lumen triple brimmed venovenous ECMO (Extracorporeal Membrane Oxygenation) cannula (1)

### PRIOR ART

Extracorporeal Membrane Oxygenation (ECMO) is a method used in patients with heart or respiratory failure in all age groups who do not respond to traditional treatment modalities. With the ECMO system, cannula or cannulas are used to remove dirty (venous) blood, and after the oxygenation of the blood is established, the arterial blood is re-injected.

Especially in pediatric age groups, cannulas used for venovenous (VV) ECMO, may have performance problems due to inadequacy of blood collection and blood supply capacity. To overcome this problem, it is necessary to use larger cannulas according to patient sizes/dimensions. As a result, there is a risk of discomfort during insertion and damage to the patient's blood vessels or heart and surrounding organs during application. In addition, an echocardiography device is used to detect the position of the cannula which was difficult to place, and the use of this device for this purpose also requires special experience. When the appropriate position is obtained in the cannula, the cannula may come off from the desired position with a small movement of the patient. In addition, cannulas that are placed in to that patients that stay in the hospital for a long time pose a risk for infection. The difficulties mentioned above limit the use of ECMO in the appropriate patients who need them, reduce the benefit of ECMO treatment, prolong hospitalization time, increase hospital deaths, increase patient mortality and increase health expenditures.

The United States patent no. US2005085761, which is incorporated in the prior art, refers to a double lumen cannula expandable for venous ECMO. This document discloses an apparatus, system and method for the use of a simple, less invasive, self-expanding, intradermal double lumen cannula group for VV ECMO. The invention described in this document provides theoretical total venous blood drainage, total extracorporeal gas exchange, and prevent recirculation and more than one (multiple) cannula use. Thus, VV ECMO is simplified, surgery and blood trauma are diminishing and its application is expanding. The cannula described in the document has three apertures in one embodiment. The dual lumen cannula of the invention also has a radiopaque marker. The cannula described in the United States patent document has a rigid structure and also has an adjustable flow mechanism. However, since this design does not include a design variety that adapts to changes in size and anatomical structure in accordance with the development of the patient, the performance of the cannula may be uncertain or under ideal. In the same design, the upper suction mouth is located very close to the spray mouth, which can cause the arterial (clean) blood to pass back through the venous (dirty) blood suction holes in the system (ie some sort of short circuit mechanism—recirculation). This can cause the total amount of oxygenated blood to be low. There is no fixing element in this design, which carries the risk of moving of cannula from where it was fixed to the skin with simple seam.

There is no structural member to prevent narrowing of the inner lumen via bowing and twisting of the cannula described in the United States patent document US2005085761. In the same design, the cannula is circular in cross-section and the structure separating the suction and ejection channels is flexible. With this design, varying lumen diameters in accordance with suction-spray power can be risky for flow stabilization. This cannula design does not have a special construction element resistant to infection. In this design, there is only one indicator showing the position of the cannula in the heart and vein. While this shows this level of cannula, it is insufficient to show the direction of openness (port).

A coaxial venous cannula is disclosed in the United States patent document U.S. Pat. No. 9,233,223, which is another prior art document. The double lumen cannula described in the document comprises two intertwined lumens wherein it has a proximal end, a distal end. In said cannula, while the first tube terminates with a multi-hole proximal end there is a second tube concentric with the first tube and continuing constantly in the middle, and there is also a distal end terminating with a drain hole therefrom.

Additionally, in the said document the location of the lumen is coaxial in the mentioned cannula and the circular structure of the cannula restricts the amount of blood that is absorbed and ejected.

In said cannula, the openings in the cannula are of a very porous structure, and it was designed this way so that every opening can perform both the suction and, the depending on the application position, can function to eject from both the neck and the head. However, this structure does not take into account the fact that the suction and ejection forces are countercurrent, so that the blood cells have a risk of causing damage during the process.

It is doubtful that placing openings at only two levels in the cannula would provide adequate blood sucking and pressure, and also it seems inevitable that this structure will be insufficient to collect venous blood from the upper and lower body of the circulatory system and direct them to a valve in a middle level. Again, in this design it is inevitable that there will be an increased short circuit—recirculation amount, which means that the performance of the cannula will be low. Also, the length of the cannula was kept long so that it can also be applied from the leg of a patient. However, this makes the use of this cannula inappropriate for short patients. For this cannula pulmonary artery placement is also recommended, however this method of placement is highly invasive (has high risk of damage to tissues), its benefit is limited and doubtful. Moreover, this practice increases the cost because it requires special experience and special equipment (such as an angio laboratory or advanced echocardiographic imaging). Again, in this type of application, a too long cannula in the heart affects the proper functioning of the heart. There is no stabilizing structure element in the same design. For this reason, there is a risk of protecting the position of the cannula. This cannula design also lacks a marker structure that indicates the position of the cannula, so a special imaging experience (echocardiography/ultrasound) is required to ensure that the cannula is correctly positioned.

### BRIEF DESCRIPTION OF THE INVENTION

The purpose of this invention is to provide a venovenous ECMO cannula that achieves performance gains by providing higher blood flow in smaller sizes and saves practitioners from the age, weight and length constraints of patients.

Another object of the invention is to achieve a venovenous ECMO cannula that facilitates the use of venovenous ECMO and reduces infant mortality in patient groups whose weight are under ten kilograms.

The other purpose of the invention is to realize a venovenous ECMO cannula which can be placed easily and correctly and the ideal settlement position is easily detected without requiring any special experience.

Another object of the invention is to realize a venovenous ECMO cannula in which loss of position of the inserted cannula with the movement of the patient's body is prevented with the use of an ear clamp in the ECMO cannula.

Another object of the invention is to realize a venovenous ECMO cannula wherein the infections risk arising from the prolonged stay in patient's body is reduced by use os a material such as silver or like.

Another object of the invention is to realize a venovenous ECMO cannula that is thin, soft and resilient but that is resistant to internal luminal contraction factors such as fracture and bending, such that the risk of vessel and cardiac damage during placement is reduced.

Another object of the invention is to realize a venovenous ECMO cannula which increases the amount of blood sucked with less suction pressure thanks to the ellipsoidal cross-sectional shape.

Another object of the invention is to realize an ECMO cannula that provides a controlled blood suction and ejection process to obtain the working system closest to the natural working order of human biology.

Another object of the invention is to realize a venovenous ECMO cannula in which the ejection opening is in the form of a wide mouth and surrounded by indicators wherein its direction is towards the cardiac valve through which the location of the venovenous ECMO cannula can be determined precisely and accurately.

Another object of the invention is to realize an ECMO cannula with four indicators wherein there are two indicators at the entrance, one at the blood ejection part and finally one at the bottom, whereby the ideal position of the cannula can be detected and verified as often as desired.

Another object of the invention is to implement a venovenous ECMO cannula having upper and lower region suction tips suitable for jugular application.

Another object of the invention is to realize a venovenous ECMO cannula in which different designs can be applied according to the patient size.

### DETAILED DESCRIPTION OF THE INVENTION

To achieve the purpose of this invention, “A Venovenous ECMO (Extracorporeal Membrane Oxygenation) Cannula” is shown in the enclosed drawings and said drawings disclose;

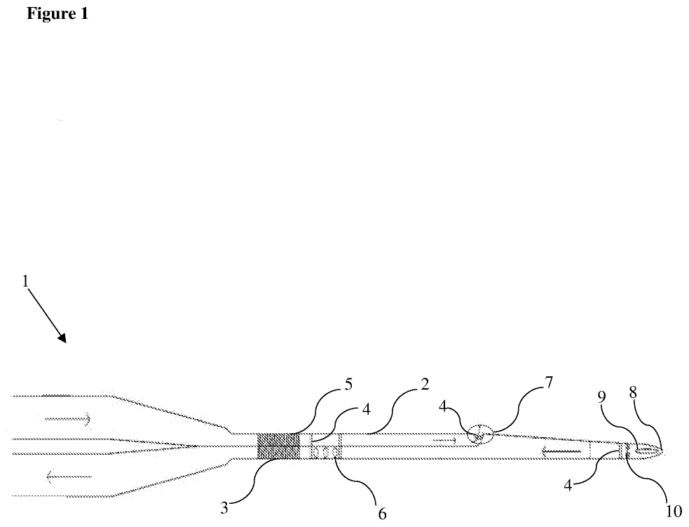

FIG. 1. Side view of the large sized cannula according to present invention.

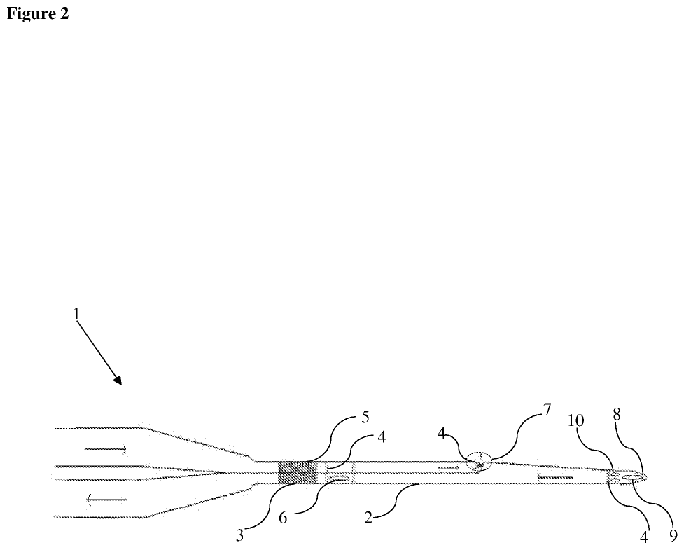

FIG. 2. Side view of the small sized cannula according to present invention.

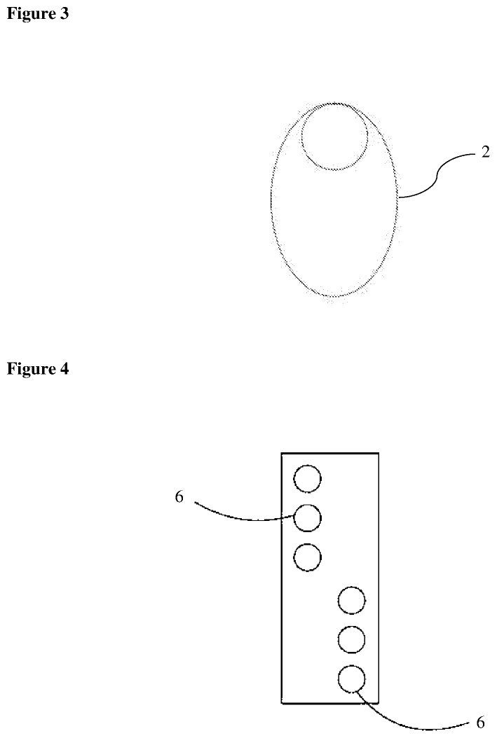

FIG. 3. A cross-sectional view of arterial and venous cannula lumens of cannulas according to present invention.

FIG. 4. A view of the upper end venous openings (venous blood inlet holes) in large sized cannula according to present invention.

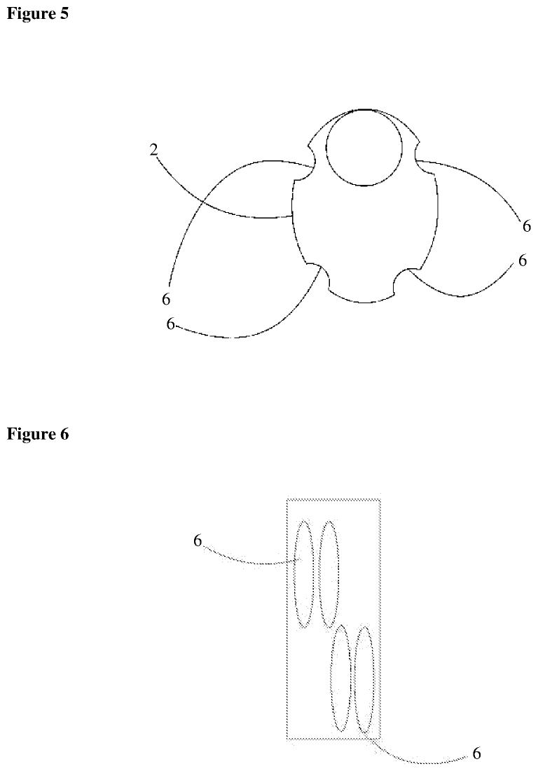

FIG. 5. A cross-sectional view of the arterial and venous lumens in the cannula of the present invention at the level of the upper venous openings (venous blood entry).

FIG. 6. A view of the upper end venous opening (venous blood entry) in cannula of the invention that is suitable for small sized patients.

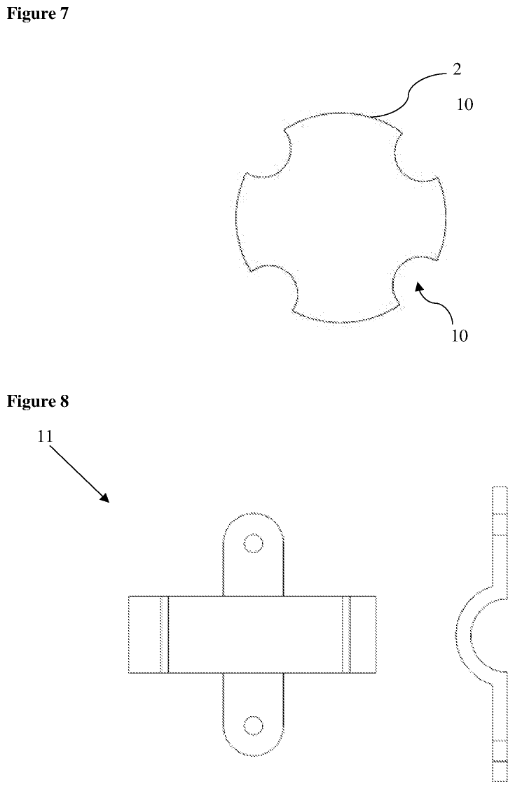

FIG. 7. The lumen cross-section view at the level of lower venous end opening of the cannula according to present invention.

FIG. 8. The detailed top view and side view of the clamp used in the cannula.

The parts in the figures are numbered individually and their correspondences are given below.
1. Cannula
2. Body
3. Coating
4. Indicator
5. Reinforced area
6. Upper venous opening
7. Arterial opening
8. Lower venous basket end
9. Basket end opening
10. Lower end hole
11. Clamp

The ECMO cannula (1) of the present invention that enables oxygenation of the venous blood and then returning said blood back to the patient, in essence comprises;
at least one body (2) having more than one openings in which the blood is collected and ejected, the body being in a double lumen structure with the ends wherein venous blood leaves and the clean blood enters,
at least one anti-bacterial coating (3) located on the upper part of the body (2) that is in contact with the patient's skin,
more than one indicator (4) that determines the level and that is located on the upper part of the body (2), in the opening wherein the blood is ejected and at the lower part of the body (2),
at least one reinforced area (5) that is resilient and resistant to breakage and located in the upper part of the body (2),
at least one upper venous opening (6) located on the body (2) into which the venous blood enters,
at least one arterial opening (7) having an indicator (4) wherein clean blood is ejected,
at least one lower venous basket end (8) located at the lower end of the body (2) and having at least one basket end opening (9),
at least one lower end hole (10) on the body (2), which is located such that is on the upper side of the lower venous basket end (8),
at least one clamp (11) securing the body (2) on the patient's skin.

In the cannula (1), the body (2) is elliptical in shape in order to increase the amount of blood sucked and is formed by non-concentric lumen placement. The lumen in the elliptical body (2) that is used to inject blood has a circular cross section.

The cannula of the invention (1) comprises two ends that perform their functions separately at the upper part of the body (2) and from which the venous blood exits and is returned back to the system after cleaning. The lumens located at the body (2), which are the continuation of the ends, extend contiguously after one point and after the arterial opening (7), and transforms into a single lumen from which the blood is sucked and stretches out to the lower part of the body (2). This way the body (2) has a structure that narrows down from upper part to lower part and has a thin lower venous basket end (8). There are preferably two indicators (4) located in the upper part of the body (2) and there is upper venous openings (6) between them and the venous blood enters the cannula from these openings. The body (2) comprises two indicators (4) located on the top and bottom of the upper venous opening (6). The arterial opening (7) from which the clean blood is ejected in on the body (2), at the lower part of the indicator (4) located under the upper venous openings (6). The anti-bacterial coating (5) is located on the body (2) between the junctions of the ends and the indicator (4) located in the upper part of the venous openings (6). The reinforced area (5) preventing breakage is located between the junction of the ends and the indicator (4) located at the upper part of the upper venous openings (6) (FIG. 1-2).

In the cannula of the present invention (1), the body (2) has an elliptical shape starting from the junction if the ends to the arterial opening (7) from which the clean blood is ejected. This leads to an increase in the surface area of the body (2) and the volume of venous lumen. A higher number of venous openings (6) that are bigger in size can be placed on the increase surface area of the body (2). The arterial lumen located in elliptical body (2) and from which the clean blood ejected has a circular section. (FIG. 3) In a preferred embodiment of the invention, the distal venous ends have a circular cross-section (FIG. 7). The lumens in the body (2) are not concentric, but have a lumen with circular cross-section in which the blood to be ejected proceeds.

In the present invention, the anti-bacterial coating (3) is preferably made of silver or to similar material. The anti-bacterial coating (3) is located on the part of the body (2) that is in contact with patient's skin preferably at the upper part of the indicator (4) on the upper part of the body (2). With the use of anti-bacterial coating (3), the risk of infecting the patient is reduced in long-term use.

In a preferred embodiment, the indicator (4) is a band made of radio opaque material. In a preferred embodiment; on the body (2) there are preferably four indicators (4). Indicators (4) located at the upper part of the body (2), at the top and bottom of upper venous openings (6) indicate the beginning and end of venous openings. The indicator (4) located just above the lower venous basket end (8) at the bottom of the body (2), is the lower radiopaque indicator of the venous lumen. On the body (2), the indicator (4) located in the arterial opening (7) is C-shaped and indicates the arterial opening. Said indicator (4) is used to direct the direction of the arterial opening (7) towards the heart valve. Indicators (4) are used to place the cannula (1) according to the natural structure of human biology and to maintain this convenient and efficient location.

In the cannula according to present invention (1), the reinforced area (5) used to prevent fracture of the body (2) is stretchable but resistant to breakage. In this way, twisting of the body (2) from the reinforced area (5) is prevented and thus the constriction or closing of the inner lumen and a decrease or stopping of the blood flow is prevented.

In a preferred embodiment of the invention, the upper venous opening (6) located at the top of the body (2) and used for entry of the venous blood can be in various shapes and numbers depending on the patient development. In the case where the patient size has increased, there are more than one round cross-sectioned upper venous openings (6) between the two indicators (4) located on the upper part of the body (2) (FIG. 6). Said upper venous openings (6) are located gradually on the body (2) (FIG. 5). In the case where the patient size is reduced, there are elliptical cross-sectioned upper venous openings (6) between the two indicators (4) located on the upper part of the body (2) (FIG. 7). Said upper venous openings (6) are elongated by extending along the body (2) and thus has an eliptical shape. This is because the amount of dirty blood coming from the upper part of the body is high because circulatory physiology changes in small-sized patients. With the design difference in small sized patients the surface area of the upper venous opening (6) is increased so that more blood is collected from the upper part of the body, thereby the performance of the cannula (1) is increased.

In the present invention, (1) the arterial opening (7) to which clean blood is ejected preferably has a round or transverse or longitudinal elliptical cross-section. In addition, the problem of hemolysis, that is, damage of blood elements by breaking, is minimized by the curved structure of the arterial opening (7) from which the blood exits.

In the cannula of the invention (1) the lower venous basket end (8) is located at the extreme end of the lower part of the body (2). On the upper part of the lower venous basket (8) there are more than one lower end holes named as lower end holes (10). In a preferred embodiment basket end openings (9) are located at the distal end of the venous lumen.

In a preferred embodiment of the invention, the number and shape of lower end hole (10) located under the body (2) and above the lower venous basket end hole (10) on the lower venous end (8) varies with respect to the size of the patient. In the case where the patient size is enlarged, there are a plurality of round or elliptic cross sectioned lower end holes (10) on top of the lower venous basket end (8) below the body (2). In the case where the patient size is smaller, there are elliptic or round shaped lower end holes (10) on top of the lower venous basket end (8) below the body (2) Said lower end holes 10 are elongated by extending along the body (2).

In the present invention, the number of the upper venous opening (6) and the lower end openings (1)0 used for suction is increased so as to increase the suction surface as a result which a cannula with a smaller body (2) provides more suction power and hence enhance the performance of the cannula (1). This way, the thinner cannula (1) provides better flow dynamics and facilitates ease of placement of the cannula (1). In the cannula according to invention(1) the upper venous opening (6) and lower end hole (10) are located on both sides of the body (2). This way, the amount of blood sucked and ejected into the system is kept in a way that does not disturb the body balance. With the help of the upper venous opening (6) and lower end hole (10) it is possible to withdraw blood from both the neck and the leg.

In the small cannula (1) designed according to the patient size, the ratio of the part comprising upper venous openings (6) located on the upper side of the body (2) and used for suction to the entire body of the cannula is more, whereas in big cannula (1) the suction part comprising lower end holes (10) located at the lower side of body (2) is more. The reason is in small children the ratio of upper body to lower body is in favor of upper body whereas as they grow up and take up adult sizes this ratio increases in favor of the lower body comprising the body and the legs.

In the cannula (1) of the present invention, the clamp (11) can be attached to and detached from the body (2) and with the help of two ears that are present on the clamp can be tied to the skin of the patient (11) with preferably a suture thread. The clamp (11) can be attached to and detached from the cannula (1) body (2) with the help of its opening and its elastic structure. Said clamp (11) comprises two channels that help fixing to the skin via winding of the suture. In a preferred embodiment of the invention the clamp (11) is used in the region that is between the reinforced area (5) and the lumen ends on the body (2).

In a preferred embodiment of the invention in the size of the body (2) designed according to the size of the patient, the distance between upper venous openings used for blood suction and lower end holes (6, 10) and the arterial openings (7) used for ejection of blood are kept in a way that would reduce the recirculation/shunt probability ratio. The cannula (1) of the invention the cross-sectioned body (2) constructs provide better flow performance. Additionally, with the cannula (1) the friction and turbulence is reduced and as a result the problem of damage to blood elements via fragmenting decreases. The possibility of varying the size of the cannula (1) according to the size of the patient not only provides increase in performance but also provides that the amount of blood sucked from the neck and leg of the patient is in accordance with the biology of the patient. The size of the cannula (1) can be changed according to the length/kg ratio and the size of the cannula (1) can be changed and also by creating a cannula (1) portfolio wherein the parameters such as cannula (1) length, arterial opening (7), upper venous opening (6), lower end hole (10) can be changed, the cannula (1) that is most suitable for the patient can be chosen. Furthermore, with the cannula (1) infant mortality is reduced by facilitating the use of venovenous ECMO in the patient group weighing less than 10 kilograms.

Around these basic concepts, it is possible to develop a wide variety of applications for the inventive subject the invention “A venovenous ECMO (Extra corporeal Membrane Oxygenation) cannula (1)”, and the invention is not limited to the examples disclosed herein, but is essentially as specified in the claims.

1. A cannula for oxygenation of contaminated blood and returning the blood to patient characterized in that said cannula comprises:
at least one body having a plurality of openings in which the blood is collected and sprayed, the body being in a double lumen structure with ends from which dirty blood leaves the body and the clean blood enters the body;
at least one anti-bacterial coating located on the upper part of the body that is in contact with the patient's skin;
more than one indicator that determines the level and that is located on the upper part of the body, in the opening wherein the blood is ejected and at the lower part of the body;
at least one reinforced area that is resilient and resistant to breakage and located in the upper part of the body;
at least one upper venous opening located on the body into which the venous blood enters;
at least one arterial opening having an indicator wherein clean blood is ejected, at least one lower venous basket end located at the lower end of the body and having at least one basket end opening;
at least one lower end hole on the body, which is located such that it is on the upper side of the lower venous basket end;
at least one clamp securing the body on the patient's skin;
a body that is elliptical in shape in order to increase the amount of blood sucked and is formed by non-concentric lumen placement wherein the lumen in the elliptical body that is used to inject blood has a circular cross section.

2. A cannula according to claim 1, characterized in that said cannula comprises a body comprising two ends connected with lumens and wherein said ends perform their functions separately at the upper part of the body and from which the venous blood exits and is returned back to the system after cleaning.

3. A cannula according to claim 1 characterized in that said cannula comprises a body comprising preferably two indicators on the upper region and there are upper venous openings in between the indicators from which the dirty blood enters.

4. A cannula according to claim 1, characterized in that said cannula comprises a body comprising two indicators located at the upper and lower part of the upper arterial openings.

5. A cannula according to claim 1, characterized in that said cannula comprises an arterial opening from which the clean blood is ejected and which is located on the body under the indicator located under the upper arterial opening.

6. A cannula according to claim 1, characterized in that said cannula comprises a bacterial coating located on the body between connection point of the ends and the indicator on top of the upper venous openings.

7. A cannula according to claim 1, characterized in that said cannula comprises a reinforced area that prevents breakage and is located between the connection point of the ends and the indicator on top of the upper venous openings.

8. A cannula according to claim 1, characterized in that said cannula comprises a body having an eliptical shape starting from the connection point of the ends until the arterial opening from which the clean blood is ejected.

9. A cannula according to claim 1, characterized in that said cannula comprises a bacterial coating made of a material such as silver or a similar material.

10. A cannula according to claim 9 characterized in that said cannula comprises an antibacterial coating located on the part of the body that is in contact with patient's skin preferably at the upper part of the indicator on the upper part of the body.

11. A cannula according to claim 1, characterized in that said cannula comprises an indicator that is a tape made up of a radio opaque material.

12. A cannula as in claim 1, characterized by indicators located at the top and bottom of the upper shed openings at the top of the body, indicating the beginning and end of openings.

13. A cannula according to claim 1, characterized in that said cannula comprises an indicator just above the lower venous basket end at the bottom of the body, that is the lower radiopaque indicator of the venous lumen.

14. A cannula according to claim 1, characterized in that said cannula comprises an indicator that is C shaped and defining the arterial opening and located on the opening on the body.

15. A cannula according to claim 1, characterized in that said cannula comprises a reinforced area that is elastic but resistant to breakage and used for preventing breakage of the body.

16. A cannula according to claim 1, characterized in that said cannula comprises an upper venous opening that is located on the top of the body and whose shape and number varies with respect to the size of the patient and which is used for introduction of contaminated blood.

17. A cannula according to claim 16, characterized in that said cannula comprises more than one round cross-sectioned upper venous openings between the two indicators located on the upper part of the body when the patient size is increased.

18. A cannula according to claim 16, characterized in that said cannula comprises upper venous openings which are located gradually on the body.

19. A cannula according to claim 16, characterized in that said cannula comprises elliptical cross-sectioned upper venous openings between the two indicators located on the upper part of the body where the patient size is reduced.

20. A cannula according to claim 19 characterized in that said cannula comprises holes that are extended through the body taking an elliptical shape.

21. A cannula according to claim 1, characterized in that said cannula comprises an arterial opening having a round or transverse or longitudinal elliptical cross-section and to which clean blood is ejected.

22. A cannula according to claim 1, characterized in that said cannula comprises arterial opening that minimizes the problem of hemolysis, that is, damage of blood elements by breaking by the curved structure of the arterial opening.

23. A cannula according to claim 1, characterized in that said cannula comprises lower venous basket end that has more than one hole, named as lower end holes on top of it.

24. A cannula according to claim 23 characterized in that said cannula comprises basket end openings located on the distal end of the venous lumen.

25. A cannula according to claim 1, wherein said cannula comprises lower end hole located under the body and above the lower venous end and whose number and shape varies with respect to the size of the patient.

26. A cannula according to claim 25, characterized in that where the patient size is enlarged, there are a plurality of round or elliptic cross sectioned lower end holes on top of the lower venous basket end below the body.

27. A cannula according to claim 25, characterized in that here the patient size is smaller, there are elliptic or round shaped lower end holes on top of the lower venous basket end below the body.

28. A cannula according to claim 1, characterized in that said cannula comprises an upper venous opening and a lower end hole that provide surface increase for blood suction so that there will be more suction power with a narrow body.

29. A cannula according to claim 1, characterized in that said cannula comprises upper venous openings and lower end holes located on both sides of the body.

30. A cannula according to claim 1, characterized in that said cannula comprise a clamp that can be attached to and detached from the body and can be tied to the skin of the patient through two ears that are present on the clamp with preferably a suture thread.

31. A cannula according to claim 30, characterized in that said cannula comprises a clamp that can be attached to and detached from the body via expanding said clamp with the help of the opening and elastic structure.

32. A cannula according to claim 30 characterized in that, said cannula comprises a clamp with two channels for stabilizing to the skin via winding of thread.

33. A cannula according to claim 1, characterized in that said cannula comprises a clamp used in the area that is between the reinforced area on the body and the lumen ends.
